(19) United States
(12) Patent Application Publication
(10) Pub. No.: US 2022/0288077 A1
(43) Pub. Date: Sep. 15, 2022

(54) PROSTATITIS TREATMENT

(71) Applicant: NATIONAL INSTITUTE OF BIOLOGICAL SCIENCES, Beijing (CN)

(72) Inventors: Zhiyuan ZHANG, Beijing (CN); Yan REN, Beijing (CN)

(73) Assignee: NATIONAL INSTITUTE OF BIOLOGICAL SCIENCES, Beijing (CN)

(21) Appl. No.: 17/636,426

(22) PCT Filed: Aug. 10, 2020
(86) PCT No.: PCT/CN2020/108085

§ 371 Date: Feb. 18, 2022

(30) Foreign Application Priority Data
Aug. 21, 2019 (CN) PCT/CN2019/101709

(51) Int. Cl. A61K 31/517; A61K 45/06; A61P 13/08

(52) U.S. Cl. CPC A61K 31/517; A61K 45/06; A61P 13/08

(57) ABSTRACT

It relates to methods of treating prostatitis comprising administering to a male in need thereof a necroptosis inhibitor, including inhibitors of RIP1, RIP3 or MLKL. It also relates to pharmaceutical composition comprising a necroptosis inhibitor and a second different drug for treating prostatitis.

### INTRODUCTION

The kinase activity of receptor-interacting kinase 1 (RIP1) is essential for both necroptosis and inflammation (Ofengeim et al., 2013). RIP1, receptor-interacting kinase 3 (RIP3) and mixed lineage kinase domain-like protein (MLKL) are key proteins in necroptosis pathway. Activation of RIP1-RIP3-MLKL cascade transmits the signals of tumor necrosis factor family of cytokines and leads cell to necroptotic death (He et al., 2009; Sun et al., 2012). Necrotic cells then release damage-associated molecular patterns (DAMPs) which can activate inflammatory responses (Pasparakis et al., 2015). RIP1 can also regulate innate immune response induced by LPS in macrophages and in mice, which requires kinase activities of both RIPK1 and RIPK3, but not the necroptosis effector protein, MLKL (Saleh et al., 2017).

It is known that seminal vesicles become enlarged as mice get old (Finch et al., 1974; Pettan-Brewer et al., 2011). Depletion of cells in the seminiferous tubules was found in aged testes. For men, aging changes in the male reproductive system may include changes in testicular tissue, enlargement of prostate (called benign prostatic hyperplasia or BPH). BPH affects about 50% of men. The prostate volume can increase from 5.5 ml in 40-49 years to 11.1 ml in 70-80 years (Fukuta et al., 2011).

The prevalence of prostatitis, including chronic prostatic inflammation (Nickel J C, 2008 Garndaglia G, 2013), ranges from 2.2 to 9.7 percent in the total male population (Krieger et al., 2008), and chronic non-bacterial (type III, according to the categories of prostatitis as defined by National Institutes of Health), called chronic prostatitis/chronic pelvic pain syndrome (CP/CPPS) accounts for about 90% prostatitis. The CP/CPPS is a common disorder of unknown etiology. Men with chronic prostatitis experience impairment in mental and physical domains of health-related quality of life (McNaughton Collins et al. 2001).

Necroptosis inhibition, such as RIP3 knockout, MLKL knockout, as well as treatment with RIPA-56, a RIP1 inhibitor can protect aged mice from seminal vesicles enlargement, loss of cells in the seminiferous tubules, decline of sperm production, and decline of reproductive capacity (Li et al., 2017); however the authors found no prostate effect: “Unlike what often happens in human upon reproductive organ aging, we did not notice any apparent anatomical difference in the anterior, dorsal, ventral, or lateral prostate.”

### SUMMARY OF THE INVENTION

We disclose that RIP1, RIP3 and MLKL inhibitors inhibit prostatitis, and demonstrate efficacy in a rat prostatitis model, providing a therapeutic application in prostatitis for RIP1, RIP3 and MLKL inhibition.

The invention provides methods and compositions for treating prostatitis or symptoms or makers thereof. In an aspect the invention provides a method of treating prostatitis comprising administering to a male in need thereof a necroptosis inhibitor.

In embodiments:

the necroptosis inhibitor is a RIP1, RIP3 or MLKL inhibitor;

the necroptosis inhibitor is a RIP1 inhibitor of Table 1, a RIP3 inhibitor of Table 2, or an MLKL inhibitor of Table 3;

the method further comprises administering to the male a second, different drug for treating prostatitis, such as selected from selective al-blockers, such as alfuzosin, doxazosin, silodosin, tamsulosin, and terazosin, 5α-reductase inhibitors such as finasteride and dutasteride, and phosphodiesterase-5 inhibitors such as sildenafil, vardenafil, and tadalafil; and/or

the method further comprises the antecedent step of diagnosis the prostatitis, and/or the subsequent step of detecting a resultant diminution or reversal of the prostatitis.

In other aspects the invention provides a pharmaceutical composition comprising a necroptosis inhibitor and a second different drug for treating prostatitis, such as selected from selective α1-blockers, such as alfuzosin, doxazosin, silodosin, tamsulosin, and terazosin, 5α-reductase inhibitors such as finasteride and dutasteride, and phosphodiesterase-5 inhibitors such as sildenafil, vardenafil, and tadalafil; and/or

the composition is in unit dosage form.

The invention encompasses all combinations of the particular embodiments recited herein.

### BRIEF DESCRIPTION OF THE DRAWINGS

FIG. 1. The histopathological examination of prostate gland. A. Sham-group. B. Carrageenan-treated model group. C. RIPA-56 (20 mg/kg, i.p., B.I.D)/Carrageenan-treated group.

### DESCRIPTION OF PARTICULAR EMBODIMENTS OF THE INVENTION

The following descriptions of particular embodiments and examples are provided by way of illustration and not by way of limitation. Those skilled in the art will recognize a variety of noncritical parameters that can be changed or modified to yield essentially similar results. All publications, patents, and patent applications cited herein, including citations therein, are hereby incorporated by reference in their entirety for all purposes. Unless contraindicated or noted otherwise, in these descriptions and throughout this application, the terms “a” and “*an” ˜ mean one or more, the term “or” means and/or.

Suitable RIP1, RIP3 and MLKL inhibitors ae known in the art, as evidenced by the following references and representative inhibitors:

TABLE 1
RIP1 inhibitors

| | |
|---|---|
| 5-((1H-indol-3-yl)methyl)-3-methyl-2-thioxoimidazolidin-4-one (Nec-1) | Nat. Chem. Biol. |
| | 2005, 1, 112-119; |
| | Bioorg. Med. |
| | Chem. Lett. 2005, |
| | 15, 5039-5044. |
| | US6756394 |
| (S)-phenyl(5-phenyl-4,5-dihydro-1H-pyrazol-1-yl)methanone | WO 2010075561 |
| 5-((1H-indol-3-yl)methyl)-3-methyl-2-thioxoimidazolidin-4-one (Nec-1s) | Cell Death Dis. |
| | 2012, 3, e437; |
| | US8741942 |
| 3-methyl-5-((7-methyl-1H-indol-3-yl)methyl)imidazolidine-2,4-dione | Nat. Chem. Biol. |
| (R)-5-((7-chloro-1H-indol-3-yl)methyl)-3-methylimidazolidine-2,4-dione | 2008, 4, 313-321; |
| | US8741942; |
| | US2011144169 |
| (R)-5-((7-chloro-1H-indol-3-yl)methyl)-3-(4-(3-(imidazo[1,2- | Cell Rep. 2015, 10, |
| b]pyridazin-3-ylethynyl)-4-methylphenyl)butyl)imidazolidine-2,4-dione | 1850-1860; |
| (Ponatinib-Nec1s) | WO2014145022; |
| | US20140323489 |
| (S)-2,2-dimethyl-1-(5-phenyl-4,5-dihydro-1H-pyrazol-1-yl)propan-1-on | Cell Death Dis. |
| (GSK963) | 2015, 1, 15009; |
| | WO2016185423 |
| (S)-2,2-dimethyl-1-(5-phenyl-4,5-dihydro-1H-pyrazol-1-yl)propan-1-one | WO2016185423 |
| (S)-1-(4-(5-phenyl-4,5-dihydro-1H-pyrazole-1-carbon yl)piperidin-1- | |
| yl)ethanone | |
| (S)-2,2-dimethyl-1-(5-(pyridin-2-yl)-4,5-dihydro-1H-pyrazol-1- | |
| yl)propan-1-one | |
| (S)-1-(4-(5-(3,5-difluorophenyl)-4,5-dihydro-1H-pyrazole-1- | |
| carbonyl)piperidin-1-yl)ethanone | |
| (S)-5-benzyl-N-(5-methyl-4-oxo-2,3,4,5- | US20170008878 |
| tetrahydrobenzo[b][1,4]oxazepin-3-yl)isoxazole-3-carboxamide | |
| (S)-5-benzyl-N-(5-methyl-4-oxo-2,3,4,5- | |
| tetrahydrobenzo[b][1,4]oxazepin-3-yl)-4H-1,2,4-triazole-3-carboxamide | |
| (S)-5-benzyl-N-(8-chloro-4-oxo-2,3,4,5- | |
| tetrahydrobenzo[b][1,4]oxazepin-3-yl)-4H-1,2,4-triazole-3-carboxamide | |
| (S)-5-benzyl-N-(5-methyl-4-oxo-7-(1H-tetrazol-5-yl)-2,3,4,5- | |
| tetrahydrobenzo[b][1,4]oxazepin-3-yl)isoxazole-3-carboxamide | |
| 8-bromo-4, 5-dihydro-1H-benzo[b]azepin-2(3H)-one | |
| (S)-5-benzyl-N-(5-methyl-4-oxo-2,3,4,5- | J. Med. Chem. |
| tetrahydrobenzo[b][1,4]oxazepin-3-yl)isoxazole-3-carboxamide | 2016, 59, 2163- |
| (GSK481) | 2178; |
| | WO2014125444 |
| (S)-5-benzyl-N-(5-methyl-4-oxo-2,3,4,5-tetrahydrobenzo[b]- | J Med Chem. 2017 |
| [1,4]oxazepin-3-yl)-1H-1,2,4-triazole-3-carboxamide (GSK2982772) | 23; 60(4): 1247- |
| | 1261; |
| | WO2016027253 |
| 1-(4-(4-aminofuro[2,3-d]pyrimidin-5-yl)phenyl)-3-(2-fluoro-5- | ACS Med. Chem. |
| (trifluoromethyl)phenyl)urea (Cpd27) | Lett. 2013, 4, |
| | 1238-1243. |
| 3-methyl-5-((7-methyl-1H-indol-3-yl)methyl)imidazolidine-2,4-dione | US8741942; |
| (R)-5-((7-chloro-1H-indol-3-yl)methyl)-3-methylimidazolidine-2,4-dione | US2011144169 |
| 3-benzyl-6,7-dihydro-3H-cyclopenta[4,5]thieno[2,3-d]pyrimidin-4(5H)- | US2012122889 |
| one | |
| N-(3-chloro-2,6-difluorobenzyl)-4-cyclopropyl-1,2,3-thiadiazole-5- | WO2009023272; |
| carboxamide | US2010317701; |
| (S)-N-(1-(2-chloro-6-fluorophenyl)ethyl)-5-cyano-1-methyl-1H-pyrrole- | US2009099242; |
| 2-carboxamide | US20120309795; |
| (S)-N-(1-(2-chloro-6-fluorophenyl)ethyl)-4-cyclopropyl-1,2,3- | US8278344; |
| thiadiazole-5-carboxamide | US9108955 |
| N-Benzyl-N-hydroxy-2,2-dimethylbutanamide | Ren et al. J Med |
| N-(4-Fluorobenzyl)-N-hydroxy-2,2-dimethylbutanamide | Chem, J. Med. |
| N-(2,4-Difluorobenzyl)-N-hydroxy-2,2-dimethylbutanamide | Chem., 2017, 60 |
| N-(3,4-Difluorobenzyl)-N-hydroxy-2,2-dimethylbutanamide | (3), pp 972-986; |
| N-Hydroxy-2,2-dimethyl-N-(2,3,4-trifluorobenzyl)butanamide | WO2016/101885 |
| N-Hydroxy-2,2-dimethyl-N-(3,4,5-trifluorobenzyl)butanamide | |
| N-Hydroxy-2,2-dimethyl-N-(2,3,5-trifluorobenzyl)butanamide | |
| (2-(3-fluorophenyl)pyrrolidin-1-yl)(1- | WO2016/101887 |
| (trifluoromethyl)cyclopentyl)methanone | |
| (2-(3-fluorophenyl)pyrrolidin-1-yl)(1- | |
| (trifluoromethyl)cyclobutyl)methanone | |
| (S)-1-(2,2-dimethylbut-3-enoyl)-4-phenylazetidin-2-one | |
| (S)-2,2-dimethyl-1-(2-phenylazetidin-1-yl)but-3-yn-1-one | |
| (S)-1-(2,2-dimethylbutanoyl)-4-phenylazetidin-2-one | |
| RIP1 inhibitors 1-151 and S1-S20, Table 1 | WO2016/101885 |
| RIP1 inhibitors 1-78 and S1-S17, Table 1 | WO2016/101887 |

TABLE 2
RIP3 inhibitors

| | |
|---|---|
| tert-butyl 2-(4-(5-(methylcarbamoyl)-1H-benzo[d]imidazol-1- | Mol. Cell 2014, 56, |
| yl)phenyl)acetate (GSK′840) | 481-495. |
| 3-(benzo[d]thiazol-5-yl)-7-(1,3-dimethyl-1H-pyrazol-5-yl)thieno[3,2- | |
| c]pyridin-4-amine (GSK′843) | |
| N-(6-(isopropylsulfonyl)quinolin-4-yl)benzo[d]thiazol-5-amine | |
| (GSK′872) | |
| N-[3-[5-(2-amino-4-pyrimidinyl)-2-(1,1-dimethylethyl)-4-thiazolyl]-2- | Cell Death Dis. |
| fluorophenyl]-2,6-difluoro-benzenesulfonamide (Dabrafenib) | 2014, 5; 5: e1278. |
| 3-(2-Imidazo[1,2-b]pyridazin-3-ylethynyl)-4-methyl-N-[4-[(4-methyl-1- | Cell Death and |
| piperazinyl)methyl]-3-(trifluoromethyl)phenyl]-benzamide (ponatinib) | Disease (2015) 6, |
| 5-[[4-[(2,3-dimethyl-2H-indazol-6-yl)methylamino]-2- | e1767. |
| pyrimidinyl]amino]-2-methyl-benzenesulfonamide (pazopanib) | |

TABLE 3
MLKL inhibitors

| | |
|---|---|
| (2E)-N-[4-[[(3-Methoxy-2-pyrazinyl)amino]sulfonyl]phenyl]-3-(5-nitro- | Cell, 2012, 148, |
| 2-thienyl)-2-propenamide (Necrosulfonamide) | 213-227; Med. |
| | Chem. Comm., |
| | 2014, 5, 333-337 |
| 1,3,7-trimethyl-8-(methylsulfonyl)-1H-purine-2,6(3H,7H)-dione(TC13-4) | Chem. Comm., |
| 58-(2,5-dimethoxybenzylsulfonyl)-1,3,7-trimethyl-1H-purine- | 2017, 53, 3637- |
| 2,6(3H,7H)-dione (TC13-58) | 3640 |
| 7-ethyl-1,3-dimethyl-8-(methylsulfonyl)-1H-purine-2,6(3H,7H)-dione | |
| (TC13-74) | |
| 1,7-dimethyl-8-(methylsulfonyl)-3-(prop-2-ynyl)-1H-purine-2,6(3H,7H)- | |
| dione (TC13-106) | |
| 2-(1,7-dimethyl-8-(methylsulfonyl)-2,6-dioxo-1H-purin-3(2H,6H,7H)- | |
| yl)acetonitrile (TC13-107) | |
| 3-(3-(3-chlorophenyl)prop-2-yn-1-yl)-8-((cyclopropylmethyl)sulfonyl)- | |
| 1,7- dimethyl-3,7-dihydro-1H-purine-2,6-dione (TC13-119) | |
| 8-((2,5-dimethoxybenzyl)sulfonyl)-1,7-dimethyl-3-(3-(2- | |
| (methylamino)pyridin-4-yl)prop-2-yn-1-yl)-3,7-dihydro-1H-purine-2,6- | |
| dione (TC13-127) | |
| 3-(3-(3-hydroxyphenyl)prop-2-yn-1-yl)-1,7-dimethyl-8-(methylsulfonyl)- | |
| 3,7-dihydro-1H-purine-2,6-dione (TC13-172) | |
| Series 1 MLKL inhibitors 1-176; Table 1 | WO2018/157800 |
| Series 2 MLKL inhibitors 1-120, Table 2 | |
| 3-((4-(methyl(4-(3-(4- | Proc Natl Acad Sci |
| (trifluoromethoxy)phenyl)ureido)phenyl)amino)pyrimidin-2- | USA, 2014, 111, |
| yl)amino)benzenesulfonamide (Compound 1) | 15072-15077; |
| | WO2015172203 |

The methods and compositions may employ the compounds in any suitable form and dosage unit, including salts, prodrugs, stereoisomers, amorphous forms, etc.

The term “pharmaceutically acceptable salts” is meant to include salts of the active compounds which are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. Prodrugs of the compounds described herein are those compounds that undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug is a compound of the invention which is administered as an ester (the “prodrug”), but then is metabolically hydrolyzed to the carboxylic acid, the active entity. Additional examples include peptidyl derivatives of a compound of the invention.

Certain compounds of the invention can exist in unsolvated forms as well as solvated forms, including hydrated forms. In general, the solvated forms are equivalent to unsolvated forms and are intended to be encompassed within the scope of the invention. Certain compounds of the invention may exist in multiple crystalline or amorphous forms. In general, all physical forms are equivalent for the uses contemplated by the invention and are intended to be within the scope of the invention.

Certain compounds of the invention possess asymmetric carbon atoms (optical centers) or double bonds; the racemates, diastereomers, geometric isomers and individual isomers are all intended to be encompassed within the scope of the invention.

The term “therapeutically effective amount” refers to the amount of the subject compound that will elicit, to some significant extent, the biological or medical response of a tissue, system, animal or human that is being sought by the researcher, veterinarian, medical doctor or other clinician, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The invention also provides pharmaceutical compositions comprising the subject compounds and a pharmaceutically acceptable excipient, particularly such compositions comprising a unit dosage of the subject compounds, particularly such compositions copackaged with instructions describing use of the composition to treat an applicable disease or condition (herein).

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Suitable excipients or carriers and methods for preparing administrable compositions are known or apparent to those skilled in the art and are described in more detail in such publications as Remington's Pharmaceutical Science, Mack Publishing Co, NJ (1991). In addition, the compounds may be advantageously used in conjunction with other therapeutic agents as described herein or otherwise known in the art, particularly other anti-necrosis agents. Hence the compositions may be administered separately, jointly, or combined in a single dosage unit.

The amount administered depends on the compound formulation, route of administration, etc. and is generally empirically determined in routine trials, and variations will necessarily occur depending on the target, the host, and the route of administration, etc. Generally, the quantity of active compound in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The compounds can be administered by a variety of methods including, but not limited to, parenteral, topical, oral, or local administration, such as by aerosol or transdermally, for prophylactic and/or therapeutic treatment. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

The therapeutics of the invention can be administered in a therapeutically effective dosage and amount, in the process of a therapeutically effective protocol for treatment of the patient. For more potent compounds, microgram (ug) amounts per kilogram of patient may be sufficient, for example, in the range of about 1, 10 or 100 ug/kg to about 0.01, 0.1, 1, 10, or 100 mg/kg of patient weight though optimal dosages are compound specific, and generally empirically determined for each compound.

In general, routine experimentation in clinical trials will determine specific ranges for optimal therapeutic effect, for each therapeutic, each administrative protocol, and administration to specific patients will also be adjusted to within effective and safe ranges depending on the patient condition and responsiveness to initial administrations. However, the ultimate administration protocol will be regulated according to the judgment of the attending clinician considering such factors as age, condition and size of the patient as well as compounds potency, severity of the disease being treated. For example, a dosage regimen of the compounds can be oral administration of from 10 mg to 2000 mg/day, preferably 10 to 1000 mg/day, more preferably 50 to 600 mg/day, in two to four (preferably two) divided doses. Intermittent therapy (e.g., one week out of three weeks or three out of four weeks) may also be used.

The subject compounds may be employed alone or in combination with other therapeutic agents. Combination therapies thus comprise the administration of at least one pharmaceutically acceptable crystalline or amorphous form of the compounds and at least one other therapeutically active agent. The subject compounds and the other therapeutically active agent(s) may be administered together in a single pharmaceutical composition or separately and, when administered separately this may occur simultaneously or sequentially in any order. The amounts of the subject compounds and the other therapeutically active agent(s) and the relative timings of administration will be selected in order to achieve the desired combined therapeutic effect. Thus in a further aspect, there is provided a combination comprising a pharmaceutically acceptable crystalline or amorphous form of the compounds together with one or more other therapeutically active agents.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Total daily dosages range from 1 mg to 2000 mg.

For use in therapy, the compounds of the invention will be normally, but not necessarily, formulated into a pharmaceutical composition, or administration unit, prior to administration to a patient. Accordingly, the invention also is directed to a pharmaceutical composition comprising a compound of the invention and one or more pharmaceutically acceptable excipients. The invention also is directed to an administration unit comprising a compound of the invention and one or more pharmaceutically acceptable excipients.

The pharmaceutical compositions or administration units of the invention may be prepared and packaged in bulk form wherein an effective amount of a compound of the invention can be extracted and then given to the patient such as with powders, syrups, and solutions for injection. Alternatively, the pharmaceutical compositions or administration units of the invention may be prepared and packaged in unit dosage form. For oral application, for example, one or more tablets or capsules may be administered. A dose of the pharmaceutical composition contains at least a therapeutically effective amount of a compound of the invention. When prepared in unit dosage form, the pharmaceutical compositions or administration units may contain from 1 mg to 1000 mg of a subject compound.

As provided herein, unit dosage forms (pharmaceutical compositions or administration units) containing from 1 mg to 1000 mg of compound may be administered one, two, three, or four times per day, preferably one, two, or three times per day, and more preferably, one or two times per day.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation. Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (supra). Accordingly, another embodiment of this invention is a method of preparing a pharmaceutical composition or administration unit comprising the step of admixing a pharmaceutically acceptable crystalline form of a subject compound with one or more pharmaceutically acceptable excipients.

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The Prostate Weight of Rat with Prostatitis was Decreased with RIPA-56 Treatment

Several animal models of chronic prostatitis have been developed for pathogenesis study and drug development, including experimental autoimmune prostatitis (EAP) models, age-related prostatitis models, hormone and castration induced prostatitis models and chemical prostatitis models (Wang & Naveed et al., 2018). Carrageenan, a polysaccharide which is commonly used to induce inflammation and subsequent pain in rodent models, can induce non-bacteria prostatitis in adult male Sprague-Dawley rats (Radhakrishnan et al., 2009; Zeng et al., 2014). The prostate got enlarged after carrageenan injection, the prostate wet weight increased from 647.8±93.7 mg to 864.9±133.2 mg in our case. The prostate index (ratio of prostate gland wet weight/body weight) of model group was 38% higher than sham-group. While treatment with a RIP1 inhibitor, RIPA-56 (Ren et al. J Med Chem. 2017 Feb. 9; 60(3):972-986) showed dose-dependent inhibition to the prostate enlargement and edema. The prostate index can decrease to 1.99±0.41 mg/g, 1.82±0.22 mg/g from 2.15±0.30 mg/g with 12.5 mg/kg and 25 mg/kg intraperitoneal treatment of RIPA-56 (B.I.D) respectively.

Finasteride, a selective inhibitor of 5α-reductase, can inhibit the conversion of testosterone to dihydrotestosterone (DHT), the primary androgen involved in normal and abnormal prostate growth. Thus finasteride is used to shrink an enlarged prostate (BPH) in adult men (Smith et al., 2009). As a control compound in our study, finasteride can decrease the prostate index of carrageenan-treated rats by 23% when orally administrated to rats at a dose of 5 mg/kg (B.I.D). Patients with chronic inflammation and BPH have been shown to both have larger prostate volumes (Mishra et al., 2007; Gandaglia et al., 2013). The inhibition of finasteride to rat prostate enlargement in the carrageenan model indicated the similarity of the rat model with human BPH and chronic prostatitis. And the efficacy of RIP1 inhibitor RIPA-56 in the rat model showed its utility to treat related prostate diseases.

The TNFα Increase in Prostate of Rat Prostatitis Model is Inhibited by RIPA-56 Treatment

Elevated cytokines in prostate fluid and semen are frequent findings in men with prostatitis (Nadler et al., 2000; He et al., 2010). It is reported that IL-1β and TNFα in prostatic secretions could be indicators in the evaluation of men with chronic prostatitis. TNF-α and IL-10 levels in expressed prostatic secretions (EPS) of chronic prostatitis patients were significantly higher in type II and type IIIa than in type IIIb and control groups. In our study, the TNFα level in the prostate homogenate of carrageenan-prostate disease model rats was significantly higher than the sham group (3.04±2.28 pg/mg vs 0.52±0.26 pg/mg prostate homogenate protein). And intraperitoneal injection of 25 mg/kg RIPA-56 (B.I.D) can decrease the TNFα level to 1.02±0.37 pg/mg dramatically.

A randomized, comparative placebo-controlled clinical study of Mercureid (MSC-428), which is a TNFα antibody, for treating chronic prostatitis indicated therapeutic efficacy—74% when applying drug Mercureid compared with placebo (therapeutic efficacy—44%) in the treatment of patients (Drannik et al., 2019), along with 57% decrease of TNFα in prostate secretion. TNFα in patient with obstructive BPH was found to be associated with a higher incidence of asymptomatic inflammatory prostatitis and prostatic calcification (Engelhardt et al., 2015). Our study implies RIP1 inhibitors, which can also decrease TNFα level in the prostate of the carrageenan-rat prostatitis model, have the potential efficacy in chronic prostatitis and BPH therapy.

White cells and neutrophils percentage in rat whole blood, and rat TNFα, PSA in serum, and IL-1β, IL-6 in prostate homogenate were also determined. However, no obvious differences were seen between sham group and model group, nor with compound-treated groups.

RIPA-56 Inhibited the Carrageenan-Induced Inflammation of Rat Prostate

Prostatitis is a polyetiological inflammation of the prostate gland, and histologically characterized by poly and mononuclear cell infiltrates (neutrophils, lymphocytes, macrophages and plasma cells) in the stromal connective tissue around the acini or ducts (Vykhovanets et al., 2007); the majority of BPH patients also had inflammatory cells (lymphocytes and macrophages) infiltrating BPH tissues. IPSS score and prostate volume showed strong correlation with the grade of prostatic inflammation (Robert et al., 2009).

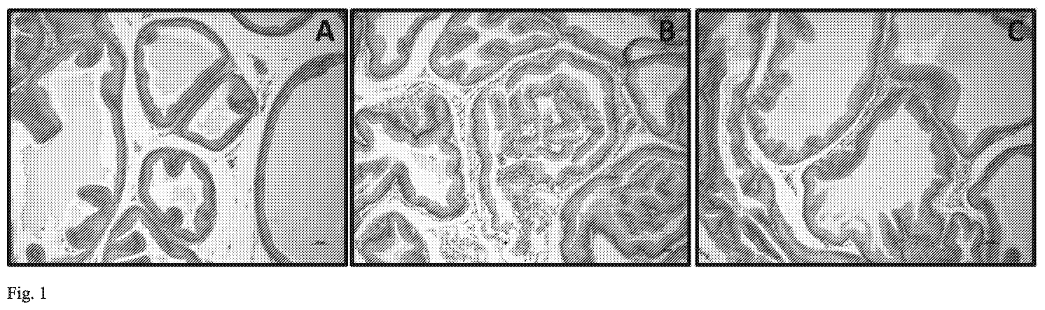

The histopathological examination of the ventral and dorsolateral lobes of prostate gland showed that intraprostatic carrageenan injection induced tissue hyperplasia, ductal ectasia, prostate papillary deformation and cell necrosis in the prostate glands, showing similar inflammation pattern to the rat model of experimental autoimmune prostatitis (EAP) (Wang et al., 2015). Increased inflammatory cells, including lymphocytes, monocytes and neutrophils diffusely accumulated in the mesenchyme (FIG. 1, B). No inflammatory cell infiltration or edema could be observed in sham group, while the glandular cavity was regular (FIG. 1, A). The inflammatory response in the prostate of RIPA-56 treated rats was less severe than the disease-model group. Only few inflammatory cells could be seen in the interstitial space (FIG. 1, C). RIPA-56 showed anti-inflammation efficacy in the carrageenan-induced prostate disease model. The alpha-blocker tamsulosin, which is used to treat the symptoms of BPH by relaxing the muscles in the prostate and bladder, also show partial anti-inflammatory efficacy in the rat EAP model (Wang et al., 2015).

Representative RIP1 Inhibitors Inhibit the Prostate Enlargement, TNFα Increasement and Prostate Inflammation.

Consistent with the RIPA-56 results, exemplary inhibitors of RIP1, including N1-urea dihydropyrazole derivatives, and N1-amide dihydropyrazole derivatives (e.g. GSK'547) (Wang et al., 2018), have similar efficacy on carrageenan-induced male rat prostate disease model, including reductions in prostate enlargement, TNFα increase and prostate inflammation. Experimental protocols for the compounds of Table 1 were based on those used for RIPA-56. Each inhibitor was intraperitoneally or orally administrated to rat before carrageenan treatment, at the dose of 20 mg/kg to 100 mg/kg. Seven days after carrageenan and inhibitor treatment, rat prostate weight, TNFα level, and prostate inflammation are all inhibited compared with carrageenan-model group.

Representative RIP3 and MLKL Inhibitors Inhibit the Prostate Enlargement, TNFα Increasement and Prostate Inflammation.

Consistent with the RIPA-56 results, exemplary inhibitors of RIP3 and MLKL have similar efficacy on carrageenan-induced male rat prostate disease model, including reductions in prostate enlargement, TNFα increase and prostate inflammation. Experimental protocols for compounds of Tables 2 and 3 were based on those used for RIPA-56. Each inhibitor was intraperitoneally or orally administrated to rat before carrageenan treatment, at the dose of 20 mg/kg to 100 mg/kg. Seven days after carrageenan and inhibitor treatment, rat prostate weight, TNFα level, and prostate inflammation are inhibited compared with carrageenan-model group.

Experimental Animals

Adult male Sprague-Dawley rats (250-350 g body weight) were used for the experiments. All animals were acclimated for 1 week before any experimental procedures.

Carrageenan-Induced Rat Prostatitis Model

All the SD rats were randomly divided into different groups (n=8-10): control group, model group, and compounds-treated groups. For injection of carrageenan or saline, rats were anesthetized with 4% chloral hydrate and were fixed in a supine position. Then, the lower abdomen above the penis of rats was shaved and the skin in this area sterilized using 3 applications of 75% medical alcohol. A small midline incision was made in the sterile area, then the bladder and the prostate carefully exposed. With a 30-gauge needle, 50 ul sterile suspension of 3% carrageenan (Sigma, MO, USA) or saline (sham group) was injected into both right and left ventral lobes of the prostate gland. Different doses of RIPA-56 (12.5 mg/kg, 25 mg/kg, IP) or Finasteride (5 mg/kg, PO) were administrated to rats before (−17 min) and after (once every 12 h) carrageenan injection. While an equal amount of solvents were intraperitoneally and orally injected to control rats.

Whole Blood and Serum Collection

Seven days after carrageenan injection, whole blood of rats was collected and analyzed for white cell counts and neutrophils percentage. Part of blood was collected into microcentrifuge tubes and allow it to clot at room temperature for 30 min then centrifuge at 3500 rpm for 10 min. The serum (supernatant) was then transferred and stored in −80° C. refrigerator until use.

Prostate Weight Assessment

Seven days after carrageenan injection, rats were sacrificed and the prostates were harvested. Ventral and dorsolateral lobes of prostate glands were removed by cutting the ducts at the urethral connection and weighed immediately after removal. The prostate index (ratio of prostate gland wet weight/body weight) was calculated.

Determination of Rat TNFα, IL-1β and IL-6 in Prostate Homogenate or Serum

Part of rat prostate were weighed, mixed with cell lysis buffer (500 μl buffer per 100 mg tissue) and homogenized using FastPrep-24 homogenizer (MP Biomedicals). The tissue homogenate was centrifuged for 10 minutes at 13,000 rpm. The supernatant was collected, and the total protein concentration was measured with the Bradford protein assay method. The samples were maintained at 4° C. during preparation. The level of cytokines was determined using rat TNFα, IL-1β and IL-6 ELISA kit (Biolegend or ProteinTech), and normalized to prostate protein concentration.

Histological Analysis

For histological analysis, one part of the prostate was fixed in 10% neutral buffered formalin for 24 h, dehydrated in ethanol, cleared in xylene, and embedded in paraffin blocks. Five micrometer sections were cut and mounted on adhesion microscope slides, and then stained with hematoxylin-eosin (H & E). Each slide was evaluated randomly in 3 different areas by an independent investigator unaware of the animal grouping using a microscope.

Active RIP1 Inhibitors
5-((1H-indol-3-yl)methyl)-3-methyl-2-thioxoimidazolidin-4-one (Nec-1)
(S)-phenyl(5-phenyl-4,5-dihydro-1H-pyrazol-1-yl)methanone
5-((1H-indol-3-yl)methyl)-3-methyl-2-thioxoimidazolidin-4-one (Nec-1s)
3-methyl-5-((7-methyl-1H-indol-3-yl)methyl)imidazolidine-2,4-dione
(R)-5-((7-chloro-1H-indol-3-yl)methyl)-3-methylimidazolidine-2,4-dione
(R)-5-((7-chloro-1H-indol-3-yl)methyl)-3-(4-(3-(imidazo[1,2-b]pyridazin-3-ylethynyl)-4-methylphenyl)butyl)imidazolidine-2,4-dione (Ponatinib-Nec1s)
(S)-2,2-dimethyl-1-(5-phenyl-4,5-dihydro-1H-pyrazol-1-yl)propan-1-on (GSK963)
(S)-2,2-dimethyl-1-(5-phenyl-4,5-dihydro-1H-pyrazol-1-yl)propan-1-one
(S)-1-(4-(5-phenyl-4,5-dihydro-1H-pyrazole-1-carbonyl)piperidin-1-yl)ethanone
(S)-2,2-dimethyl-1-(5-(pyridin-2-yl)-4,5-dihydro-1H-pyrazol-1-yl)propan-1-one
(S)-1-(4-(5-(3,5-difluorophenyl)-4,5-dihydro-1H-pyrazole-1-carbonyl)piperidin-1-yl)ethanone
(S)-5-benzyl-N-(5-methyl-4-oxo-2,3,4,5-tetrahydrobenzo[b][1,4]oxazepin-3-yl)isoxazole-3-carboxamide
(S)-5-benzyl-N-(5-methyl-4-oxo-2,3,4,5-tetrahydrobenzo[b][1,4]oxazepin-3-yl)-4H-1,2,4-triazole-3-carboxamide
(S)-5-benzyl-N-(8-chloro-4-oxo-2,3,4,5-tetrahydrobenzo[b][1,4]oxazepin-3-yl)-4H-1,2,4-triazole-3-carboxamide
(S)-5-benzyl-N-(5-methyl-4-oxo-7-(1H-tetrazol-5-yl)-2,3,4,5-tetrahydrobenzo[b][1,4]oxazepin-3-yl)isoxazole-3-carboxamide
8-bromo-4, 5-dihydro-1H-benzo[b]azepin-2(3H)-one
(S)-5-benzyl-N-(5-methyl-4-oxo-2,3,4,5-tetrahydrobenzo[b][1,4]oxazepin-3-yl)isoxazole-3-carboxamide (GSK481)
(S)-5-benzyl-N-(5-methyl-4-oxo-2,3,4,5-tetrahydrobenzo[b]-[1,4]oxazepin-3-yl)-1H-1,2,4-triazole-3-carboxamide (GSK2982772)
1-(4-(4-aminofuro[2,3-d]pyrimidin-5-yl)phenyl)-3-(2-fluoro-5-(trifluoromethyl)phenyl)urea (Cpd27)
3-methyl-5-((7-methyl-1H-indol-3-yl)methyl)imidazolidine-2,4-dione
(R)-5-((7-chloro-1H-indol-3-yl)methyl)-3-methylimidazolidine-2,4-dione
3-benzyl-6,7-dihydro-3H-cyclopenta[4,5]thieno[2,3-d]pyrimidin-4(5H)-one
N-(3-chloro-2,6-difluorobenzyl)-4-cyclopropyl-1,2,3-thiadiazole-5-carboxamide
(S)—N-(1-(2-chloro-6-fluorophenyl)ethyl)-5-cyano-1-methyl-1H-pyrrole-2-carboxamide
(S)—N-(1-(2-chloro-6-fluorophenyl)ethyl)-4-cyclopropyl-1,2,3-thiadiazole-5-carboxamide
N-Benzyl-N-hydroxy-2,2-dimethylbutanamide
N-(4-Fluorobenzyl)-N-hydroxy-2,2-dimethylbutanamide
N-(2,4-Difluorobenzyl)-N-hydroxy-2,2-dimethylbutanamide
N-(3,4-Difluorobenzyl)-N-hydroxy-2,2-dimethylbutanamide
N-Hydroxy-2,2-dimethyl-N-(2,3,4-trifluorobenzyl)butanamide
N-Hydroxy-2,2-dimethyl-N-(3,4,5-trifluorobenzyl)butanamide
N-Hydroxy-2,2-dimethyl-N-(2,3,5-trifluorobenzyl)butanamide
(2-(3-fluorophenyl)pyrrolidin-1-yl)(1-(trifluoromethyl)cyclopentyl)methanone
(2-(3-fluorophenyl)pyrrolidin-1-yl)(1-(trifluoromethyl)cyclobutyl)methanone
(S)-1-(2,2-dimethylbut-3-enoyl)-4-phenylazetidin-2-one
(S)-2,2-dimethyl-1-(2-phenylazetidin-1-yl)but-3-yn-1-one
(S)-1-(2,2-dimethylbutanoyl)-4-phenylazetidin-2-one,
RIP1 inhibitors 1-151 and S1-S20, Table 1; WO2016/101885
RIP1 inhibitors 1-78 and S1-S17, Table 1; WO2016/101887

Active RIP3 Inhibitors
tert-butyl 2-(4-(5-(methylcarbamoyl)-1H-benzo[d]imidazol-1-yl)phenyl)acetate (GSK'840)
3-(benzo[d]thiazol-5-yl)-7-(1,3-dimethyl-1H-pyrazol-5-yl)thieno[3,2-c]pyridin-4-amine (GSK'843)
N-(6-(isopropylsulfonyl)quinolin-4-yl)benzo[d]thiazol-5-amine (GSK'872)
N-[3-[5-(2-amino-4-pyrimidinyl)-2-(1,1-dimethylethyl)-4-thiazolyl]-2-fluorophenyl]-2,6-difluoro-benzenesulfonamide (Dabrafenib)
3-(2-Imidazo[1,2-b]pyridazin-3-ylethynyl)-4-methyl-N-[4-[(4-methyl-1-piperazinyl)methyl]-3-(trifluoromethyl)phenyl]-benzamide (ponatinib)
5-[[4-[(2,3-dimethyl-2H-indazol-6-yl)methylamino]-2-pyrimidinyl]amino]-2-methyl-benzenesulfonamide (pazopanib)

Active MLKL Inhibitors
(2E)-N-[4-[[(3-Methoxy-2-pyrazinyl)amino]sulfonyl]phenyl]-3-(5-nitro-2-thienyl)-2-propenamide (Necrosulfonamide)
1,3,7-trimethyl-8-(methylsulfonyl)-1H-purine-2,6(3H,7H)-dione (TC13-4)
(2,5-dimethoxybenzylsulfonyl)-1,3,7-trimethyl-1H-purine-2,6(3H,7H)-dione (TC13-58)
7-ethyl-1,3-dimethyl-8-(methylsulfonyl)-1H-purine-2,6(3H,7H)-dione (TC13-74)
1,7-dimethyl-8-(methylsulfonyl)-3-(prop-2-ynyl)-1H-purine-2,6(3H,7H)-dione (TC13-106)
2-(1,7-dimethyl-8-(methylsulfonyl)-2,6-dioxo-1H-purin-3(2H,6H,7H)-yl)acetonitrile (TC13-107)
3-(3-(3-chlorophenyl)prop-2-yn-1-yl)-8-((cyclopropylmethyl)sulfonyl)-1,7-dimethyl-3,7-dihydro-1H-purine-2,6-dione (TC13-119)
8-((2,5-dimethoxybenzyl)sulfonyl)-1,7-dimethyl-3-(3-(2-(methylamino)pyridin-4-yl)prop-2-yn-1-yl)-3,7-dihydro-1H-purine-2,6-dione (TC13-127)
3-(3-(3-hydroxyphenyl)prop-2-yn-1-yl)-1,7-dimethyl-8-(methylsulfonyl)-3,7-dihydro-1H-purine-2,6-dione (TC13-172)
3-((4-(methyl(4-(3-(4-(trifluoromethoxy)phenyl)ureido)phenyl)amino)pyrimidin-2-yl)amino)benzenesulfonamide (Compound 1)
Series 1 MLKL inhibitors 1-176; Table 1; Series 2 MLKL inhibitors 1-120, Table 2;
WO2018/157800.

### REFERENCES

Drannik, G, Gorpinchenko, I., Kurchenko, A. and Gusev, S. (2019) “Efficacy study of new drug Mercureid (MSC-428) in anti-TNFα therapy for chronic prostatitis”. Acta Scientific Cancer Biology 3(5), 09-16.
Engelhardt, P., Seklehner, S., Brustmann, H., Riedl, C., Lusuardi, L. (2015) Tumor necrosis factor-α expression in patients with obstructive benign prostatic hyperplasia is associated with a higher incidence of asymptomatic inflammatory prostatitis NIH category IV and prostatic calcificationScand J Urol. 49(6), 472-478.
Finch, C. E., and Girgis, F. G. (1974). Enlarged seminal vesicles of senescent C57BL-6J mice. J Gerontol 29, 134-138.
Fukuta, F., Masumori, N., Mori, M. and Tsukamoto T. (2011) Internal prostatic architecture on transrectal ultrasonography predicts future prostatic growth natural history of prostatic hyperplasia in a 15-year longitudinal community-based study. Prostate 71, 597-603.
Gandaglia, G., Briganti, A., Gontero, P., Mondaini, N., Novara, G., Salonia, A., Sciarra, A. and Montorsi, F. (2013) The role of chronic prostatic inflammation in the pathogenesis and progression of benign prostatic hyperplasia (BPH). BJU Int. 112, 432-441.
He, L., Wang, Y., Long, Z. and Jiang, C. (2010) Clinical significance of IL-2, IL-10, and TNF- in prostatic secretion of patients with chronic prostatitis. Urology 75, 654-657.
He, S., Wang, L., Miao, L., Wang, T., Du, F., Zhao, L., Wang, X. (2009) Receptor interacting protein kinase-3 determines cellular necrotic response to TNF-alpha. Cell 137(6), 1100-1111.
Krieger, J., Lee, S., Jeon, J., Cheah, P., Liong, M. and Riley, D. (2008) Epidemiology of prostatitis. Int J Antimicrob Agents 31(suppl 1), S85-S90.
Li, D., Meng, L., Xu, T., Su, Y., Liu, X., Zhang, Z., and Wang, X. (2017) RIPK1-RIPK3-MLKL-dependent necrosis promotes the aging of mouse male reproductive system. eLife 6, e27692.
McNaughton Collins, M., Pontari, M., O'Leary, M., Calhoun, E., Santanna, J., Landis, J., Kusek, J. and Litwin, M. (2001) Quality of life is impaired in men with chronic prostatitis: the Chronic Prostatitis Collaborative Research Network. J Gen Intern Med. 16(10), 656-662.
Mishra, V. C., Allen, D. J., Nicolaou, C. Sharif, H., Hudd, C., Karim, O. M. A., Motiwala, H. G. and Laniado, M. E. (2007) Does intraprostatic inflammation have a role in the pathogenesis and progression of benign prostatic hyperplasia? BJU Int. 100, 327-331.
Nadler, R., Koch, A., Calhoun, E., Campbell, P., Pruden, D., Bennett, C., Yarnold, P. and Schaeffer, A. (2000) IL-1beta and TNF-alpha in prostatic secretions are indicators in the evaluation of men with chronic prostatitis. J Urol. 164(1), 214-218.
Nickel. J., Roehrborn, C., O'Leary. M., Bostwick, D., Somerville. M. and Rittmaster, R. (2008) The relationship between prostate inflammation and lower urinary tract symptoms: examination of baseline data from the REDUCE trial. Eur Urol 54, 1379-1384.
Ofengeim. D. and Yuan, J. (2013) Regulation of RIP1 kinase signalling at the crossroads of inflammation and cell death. Nat. Rev. Mol. Cell Biol. 14, 727-736.
Pasparakis, M. and Vandenabeele, P. (2015) Necroptosis and its role in inflammation. Nature 517, 311-320.
Pettan-Brewer, C., and Treuting, P.M. (2011). Practical pathology of aging mice. Pathobiol Aging Age Relat Dis. 1.
Radhakrishnan, R., Nallu, R. (2009) Development and characterisation of a novel animal model of prostate inflammation-induced chronic pelvic pain. Inflammopharmacology 17(1), 23-28.
Robert, G., Descazeaud, A., Nicolaiew, N., Terry, S., Sirab, N., Vacherot, F., Maillé, P., Allory, Y. and Taille, A. (2009) Inflammation in benign prostatic hyperplasia: a 282 patients' immunohistochemical analysis. Prostate 69 (16), 1774-1780.
Saleh, D., Najjar, M., Zelic, M., Shah, S., Nogusa, S., Polykratis, A., Paczosa, M., Gough, P., Bertin, J., Whalen, M., et al. (2017) Kinase Activities of RIPK1 and RIPK3 Can Direct IFN-β Synthesis Induced by Lipopolysaccharide. J Immunol. 198(11), 4435-4447.
Smith, A. and Carson, C. (2009) Finasteride in the treatment of patients with benign prostatic hyperplasia: a review. Ther Clin Risk Manag. 5, 535-545.
Sun, L., Wang, H., Wang, Z., He, S., Chen, S., Liao, D., Wang, L., Yan, J., Liu, W., Lei, X., Wang, X. (2012) Mixed lineage kinase domain-like protein mediates necrosis signaling downstream of RIP3 kinase. Cell 148 (1-2), 213-227.
Vykhovanets, E., Resnick, M., MacLennan, G. and Gupta, S. (2007) Experimental rodent models of prostatitis: limitations and potential. Prostate Cancer and Prostatic Diseases 10, 15-29.
Wang, W., Marinis, J., Beal, A., Savadkar, S., Wu, Y., Khan, M., Taunk, P., Wu, N., Su, W., Wu, J., et al. (2018) RIP1 kinase drives macrophage-mediated adaptive immune tolerance in pancreatic cancer. Cancer Cell 34 (5), 757-774.
Wang, W., Naveed, M., Baig, M., Abbas, M. and Zhou, X. (2018) Experimental rodent models of chronic prostatitis and evaluation criteria. Biomedicine & Pharmacotherapy 108, 1894-1901
Wang, X., Zhong, S., Xu, T., Xia, L., Zhang, X., Zhu, Z., Zhang, M. and Shen, Z. (2015) Histopathological classification criteria of rat model of chronic prostatitis/chronic pelvic pain syndrome. Int Urol Nephrol. 47(2), 307-316.
Zeng, F., Chen, H., Yang, J., Wang, L., Cui, Y., Guan, X., Wang, Z., Niu, J., Zu, X., Qi, L., Zhang, X., Tang, Z., and Liu, L. (2014) Development and validation of an animal model of prostate inflammation-induced chronic pelvic pain: evaluating from inflammation of the prostate to pain behavioral modifications. PLoS ONE 9(5), e96824.

1. A method of treating prostatitis, comprising administering to a male in need thereof a necroptosis inhibitor.

2. The method of claim 1 wherein the necroptosis inhibitor is a RIP1, RIP3 or MLKL inhibitor.

3. The method of claim 1 wherein the necroptosis inhibitor is a RIP1 inhibitor of Table 1.

4. The method of claim 1 wherein the necroptosis inhibitor is a RIP3 inhibitor of Table 2.

5. The method of claim 1 wherein the necroptosis inhibitor is a MLKL inhibitor of Table 3.

6. The method of claim 1, wherein the method further comprises administering to the male a second, different drug for treating prostatitis.

7. The method of claim 1, wherein the method further comprises administering to the male a second, different drug for treating prostatitis, selected from alfuzosin, doxazosin, silodosin, tamsulosin, terazosin, finasteride, dutasteride, sildenafil, vardenafil, and tadalafil.

8. The method of claim 1, wherein the method further comprises an antecedent step of diagnosing the prostatitis, and/or a subsequent step of detecting a resultant diminution or reversal of the prostatitis.

9. A pharmaceutical composition comprising a necroptosis inhibitor and a second different drug for treating prostatitis.

10. The pharmaceutical composition of claim 9, wherein the different drug is selected from alfuzosin, doxazosin, silodosin, tamsulosin, terazosin, finasteride, dutasteride, as sildenafil, vardenafil, and tadalafil.

11. The composition of claim 9 in unit dosage form.

12. The method of claim 3, wherein the method further comprises administering to the male a second, different drug for treating prostatitis.

13. The method of claim 4, wherein the method further comprises administering to the male a second, different drug for treating prostatitis.

14. The method of claim 5, wherein the method further comprises administering to the male a second, different drug for treating prostatitis.

15. The method of claim 3, wherein the method further comprises administering to the male a second, different drug for treating prostatitis, selected from selective α1-blockers, 5α-reductase inhibitors, and phosphodiesterase-5 inhibitors.

16. The method of claim 4, wherein the method further comprises administering to the male a second, different drug for treating prostatitis, selected from selective α1-blockers, 5α-reductase inhibitors, and phosphodiesterase-5 inhibitors.

17. The method of claim 5, wherein the method further comprises administering to the male a second, different drug for treating prostatitis, selected from selective α1-blockers, 5α-reductase inhibitors, and phosphodiesterase-5 inhibitors.

18. The method of claim 3, wherein the method further comprises an antecedent step of diagnosing the prostatitis, and/or a subsequent step of detecting a resultant diminution or reversal of the prostatitis.

19. The method of claim 4, wherein the method further comprises an antecedent step of diagnosing the prostatitis, and/or a subsequent step of detecting a resultant diminution or reversal of the prostatitis.

20. The method of claim 5, wherein the method further comprises an antecedent step of diagnosing the prostatitis, and/or a subsequent step of detecting a resultant diminution or reversal of the prostatitis.
